(19) United States
(12) Patent Application Publication
(10) Pub. No.: US 2007/0118197 A1
(43) Pub. Date: May 24, 2007

(54) Probe for Identifying Injection Site for Deep Brain Neural Prostheses

(75) Inventor: Gerald E. Loeb, South Pasadena, CA (US)

Correspondence Address: MCDERMOTT WILL & EMERY LLP, 2049 CENTURY PARK EAST, 34TH FLOOR, LOS ANGELES, CA, 90067-3208, US

(73) Assignee: Alfred E. Mann Institute for Biomedical Engineering at the University of Southern Californ

(21) Appl. No.: 11/457,004

(22) Filed: Jul. 12, 2006

Related U.S. Application Data
(60) Provisional application No. 60/698,314, filed on Jul. 12, 2005.

(51) Int. Cl. A61N 1/05

(52) U.S. Cl. 607/116; 607/45

(57) ABSTRACT

Devices and systems for recording and/or stimulating electrical signals in order to identify a target site within a patient's brain for further electrical stimulation and chemical treatments of the brain. The deep brain stimulation devices and methods include implantable devices having various microelectrode configurations and drug delivery mechanisms. The devices can be used to treat a variety of neurological conditions.

### CROSS REFERENCE TO RELATED APPLICATIONS

This United States Patent Application is related to and claims the benefit of the filing date of U.S. Provisional Patent Application Ser. No. 60/698,314, filed Jul. 12, 2005, entitled “Deep Brain Neural Prosthetic System,” attorney docket no. 64693-137, the contents of which are incorporated herein by reference. This United States Patent Application is also related to co-pending U.S. patent application Ser. No. 11/456,950, which is being filed contemporaneously on Jul. 12, 2006, entitled “Deep Brain Neural Prosthetic System,” inventors Gerald E. Loeb and Hagai Bergman, attorney docket no. 64693-165, the contents of which are also incorporated herein by reference.

### BACKGROUND

1. Field

This application relates generally to devices and systems for providing electrical and chemical treatments to the brain.

2. Description of Related Art

Deep brain stimulation has become well-accepted clinically and successful commercially for the treatment of various symptoms of Parkinson's disease. It is usually prescribed after systemic pharmacological treatment to restore dopamine levels becomes ineffective or unacceptable because of side effects. Its use is expanding into related motor disorders arising from dysfunction of the basal ganglia. Potential applications include a wide range of clinical neuroses such as depression, obsessive-compulsive disorder, obesity, and other addictive disorders.

One limitation of deep brain stimulation has been the complexity of chemical and electrical circuitry in the basal ganglia (BG), a small structure (˜2-3 cm egg) located deep in the midbrain. Both stereotaxic and neurophysiological recording techniques are currently used to insert a four contact electrode into the BG on one or both brain hemispheres. Stimulation of the wrong site can produce poor results, including severe side effects. Penetration required to identify the correct target can produce neural damage along the track and risks extensive damage from bleeding. Continuous stimulation appears to disrupt rather than to repair pathological activity, which is likely to cause its own functional deficits, perhaps related to learning new skills. Local administration of dopamine within the BG could avoid many of the side effects of systemic administration and could potentiate the therapeutic effects of electrical stimulation, perhaps improving outcomes and prolonging the period of time for which progressively degenerative BG diseases can be successfully treated.

### SUMMARY

This application presents neural prosthetic systems for deep brain stimulation that can be directed more specifically, programmed more flexibly, used for a longer period of time and integrated with various chemical therapies.

It is understood that other embodiments of the devices and methods will become readily apparent to those skilled in the art from the following detailed description, wherein it is shown and described only exemplary embodiments of the devices, methods and systems by way of illustration. As will be realized, the devices, systems and systems are capable of other and different embodiments and its several details are capable of modification in various other respects, all without departing from the spirit and scope of the invention. Accordingly, the drawings and detailed description are to be regarded as illustrative in nature and not as restrictive.

### BRIEF DESCRIPTION OF THE DRAWINGS

Aspects of the microstimulator injection devices and systems are illustrated by way of example, and not by way of limitation, in the accompanying drawings, wherein:

FIG. 1 is a side cross-sectional illustration of an exemplary deep brain neural prosthetic system; and

FIG. 2 is a schematic illustration an exemplary deep brain neural prosthetic system.

### DETAILED DESCRIPTION

The detailed description set forth below in connection with the appended drawings is intended as a description of exemplary embodiments and is not intended to represent the only embodiments in which the deep brain stimulation devices, methods and systems can be practiced. The term “exemplary” used throughout this description means “serving as an example, instance, or illustration,” and should not necessarily be construed as preferred or advantageous over other embodiments. The detailed description includes specific details for the purpose of providing a thorough understanding of the deep brain stimulation devices, methods and systems. However, it will be apparent to those skilled in the art that the deep brain stimulation devices, methods and systems may be practiced without these specific details.

The deep brain stimulation devices and methods include implantable devices having various microelectrode configurations and drug delivery mechanisms. The devices can be used to treat a variety of neurological conditions. For example, various applications that may be achieved with the present devices are described in the following articles, which are incorporated by reference: Kitagawa, M., Murata, J., Kikuchi, S., Sawamura, Y., Saito, H., Sasaki, H., & Tashiro, K. (2000), “Deep brain stimulation of subthalamic area for severe proximal tremor,” Neurology, 55(1), 114-116; Kumar, R., Dagher, A., Hutchinson, W. D., Lang, A. E., & Lozano, A. M. (1999), “Globus pallidus deep brain stimulation for generalized dystonia: clinical and PET investigation,” Neurology, 53(4), 871-874; Phillips, N. I., & Bhakta, B. B. (2000), “Effect of deep brain stimulation on limb paresis after stroke,” Lancet, 356(9225), 222-223; Taira, T., Kawamura, H., & Takakura, K. (1998), “Posterior occipital approach in deep brain stimulation for both pain and involuntary movement. A case report,” Stereotact Funct Neurosurg, 70(1), 52-56; Tasker, R. R., & Vilela Filho, O. (1995), “Deep brain stimulation for neuropathic pain,” Stereotact Funct Neurosurg, 65(1-4), 122-124.

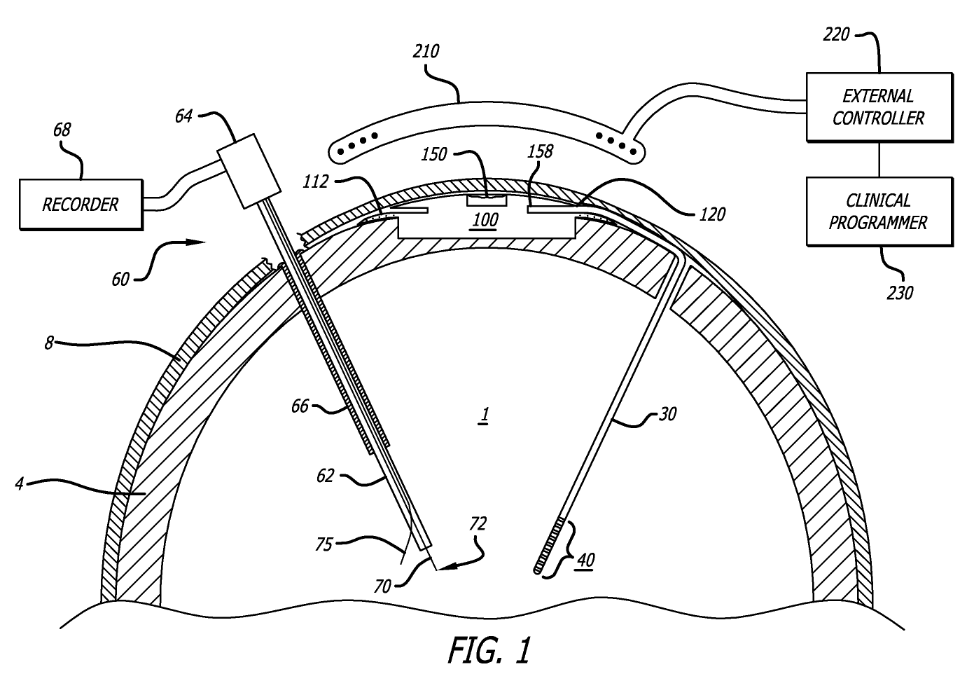

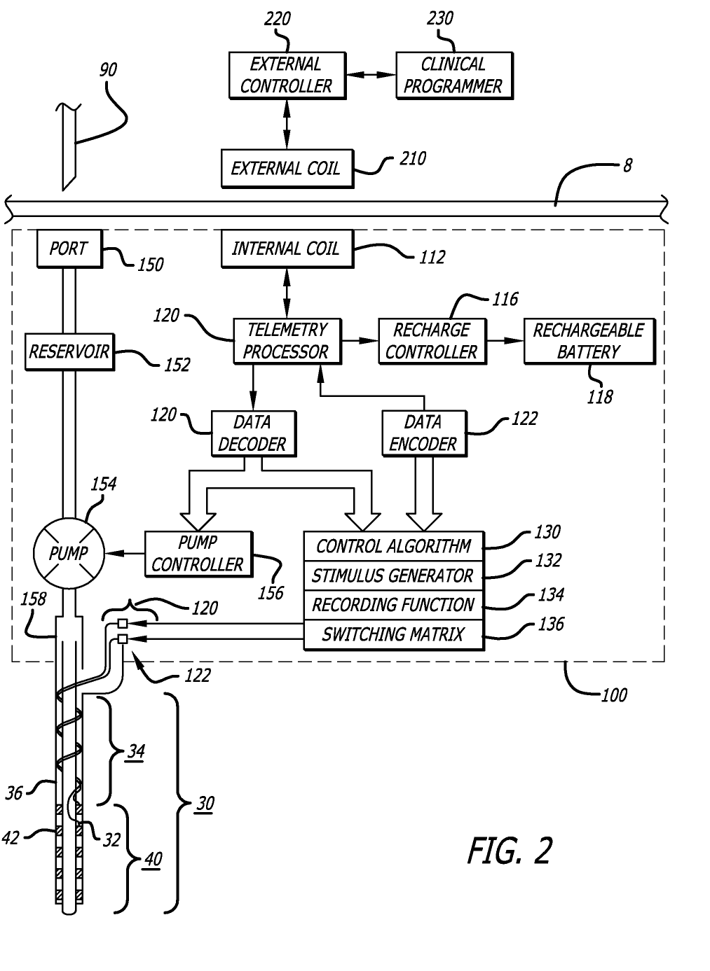

The device includes a thin electrode array (about 1-2 mm diameter) with 4-8 contacts on 1-2 mm centers plus a central lumen for drug infusion from a fully implanted pump with refillable reservoir. A single electronics and pump module with connections to two electrode arrays could be small enough to locate under the scalp. FIG. 1 provides a mechanical cross-section showing all major components. FIG. 2 provides a functional block diagram of the chronically implanted system.

FIG. 1 shows a probe 60 with two microelectrodes within a hollow guide tube 66: a fixed, straight microelectrode 70 that advances with the probe 60 and a curved, lateral microelectrode 75 that can be independently moved by advancer 64 so as to extend laterally on an arc away from the central track. The direction of the extension can depend on axial rotation of the probe 60 in the guide tube 66. Both electrodes may be made of pure iridium metal with laser-exposed insulation composed of any of the polymers of polyparaxylylene (commonly trademarked as Parylene), as described in U.S. Pat. No. 5,524,338, incorporated herein by reference. This combination of materials can be used safely to apply stimuli at therapeutic levels without degrading their single unit recording capabilities. These materials also have the requisite springiness (i.e. elasticity) and durability to survive multiple cycles of straightening when the curved lateral microelectrode 75 is pulled into the lumen of the guide tube (66), followed by reforming of curvature when extended from the guide tube 66.

Referring also to FIG. 2, the electrode contacts 42 that make up the interface region 40 of the implanted array 30 can be made from thin-wall rings of sintered Ta stacked with polymeric spacing rings to form a relatively rigid distal segment with a hollow core through which the Ta leads and drug infusion can pass. The central core may be built around a thin-walled flexible tubing such as polyimide, with laser-drilled perforations at the levels of the electrode contacts 42 to permit egress of the drug being infused via pump 154. The proximal part of the shaft and leads functions as a cable 34, which may be made of silicone elastomer molded around a multifilar spiral for the electrode leads with a central hollow core. This core may accommodate a stiffening stylus during implantation, which can be removed to leave the lumen for drug infusion. The drug passes through and may be diffused by the sintered Ta electrode contacts 42, which can be a sponge-like structure with continuous pores that are too fine to be clogged by connective tissue, typically 5p or less pore size. By making both the leads 32 and electrode contacts 42 from pure tantalum metal, they may be anodized to provide an integral insulation and capacitive coupling for the stimulation. Such electrode materials also provide frequency response down to the 2 Hz low-cutoff of the evoked potentials that may be detected by recording function 134 from one or more electrode contacts 42 selected by switching matrix 136. An all-tantalum electrode and lead system that can be used is described in U.S. Pat. No. 5,833,714, which is incorporated herein by reference. The drug solution may have a low enough ionic content so that it does not significantly shunt the electrodes, which can be used independently to stimulate and record from selectable sites along the distal shaft.

A single titanium case may contain all electronic components of the implanted controller 100 except for the one or two implanted arrays 30 and their associated connectors 120 and an RF internal coil 112 that surrounds the hermetic case or can be attached as a satellite in the manner of cochlear implants. The RF coil can be used for inductive coupling to an external coil 210 in order to recharge an internal, rechargeable battery 118 and for bidirectional data transmission to query and program the electronic functions. In normal operation, the system may work autonomously according to a control algorithm 130, with only simple on-off and perhaps state commands transmitted from a patient-operated remote control.

Each electrode may be switchable to record or stimulate. There may be 4-8 independently programmable sources of bipolar stimulation that could be combined to provide steerable stimulation fields. Recordings can be low frequency field potentials (2-70 Hz) from a low impedance (˜1 kΩ), low amplitude (˜100 μV) source, in some examples no more than one channel per array. The signal may be digitized and processed to detect energy in various frequency bands, which could trigger state changes in stimulation or drug delivery according to control algorithm 130. The stimulation may be timed to temporal details of the recorded signal. A data logging capacity may be included that could be transmitted between the internal coil 112 and the external coil 210 and hence to the clinical programmer 230 via the data encoder 122 and telemetry processor 114 when the patient is seen in the clinic. In some embodiments individual contacts in each array may be more or less permanently assigned during the postoperative fitting and programming period to record and/or stimulate.

Conventional pacemaker technology may be employed for encasing implanted controller 100. For example, a thin wall, drawn titanium case with laser or electron-beam welded feedthroughs and seals may be utilized. Given an appropriate curvature, a fairly large diameter may be used under the scalp at midline. Some portion may be recessed partially into the skull to provide adequate vertical height and anchoring.

The electrodes may be detachable from the electronics package, due to variable skull size and approach angles to the BG. In some embodiments, the electronics may be replaced without dislodging electrodes. If the central lumen is used for a stiffening trochar during insertion, the lumen may be able to self-seal or be sealed after removal to prevent leakage of unfused drug. It is generally necessary for the entire connector 120 for the implanted array 30, including both its fluidic coupling 158 and connector contacts 122 to be designed so as to have an outside diameter no greater than the outside diameter of cable 34 and any jacket 36 encasing it and small than the inside diameter of guide tube 66, which must be removed by passing it over the implanted array 30 after its interface 40 is correctly located in the BG. This can be achieved by circumferential band-shape for connector contacts 122 such as are commonly employed in spinal cord electrode arrays that are inserted similarly through a guide tube, and elastomeric gaskets for coupling 158 such as are commonly employed in intrathecal drug pumps whose catheters are inserted similarly through a guide tube.

The deep brain stimulation devices may control the release of neurotransmitters such as dopamine into the BG around the electrode sites. The release may be fairly diffuse to avoid toxic local doses and it may be modulated over a range of about 0.2-10× baseline. Baseline release tends to occur for 1-5 seconds, followed by a peak or valley lasting about 0.2-1 s. A control algorithm 130 could trigger these releases according to field potentials recorded by electrode contacts 42 in the BG (see, for example, discussion of closed-loop control below). Local injection may avoid the blood-brain barrier, high dosages and side-effects of systemically administered drugs.

The device may employ multiple, closely spaced and independently controllable electrode contacts so that stimulation can be adjusted after the electrode is fixed in place. The device may provide therapeutic stimulation parameters such as 200-500 μA×100 μs@160 pps. Stimulation and drug delivery may be gated and modulated according to oscillatory field potentials that could be recordable by selected contacts in the array. Single unit potentials are normally used to guide initial placement (see below), but recording them chronically would be problematic. During normal function, the BG has relatively continuous and asynchronous activity that produces little or no coherent field potentials. In a pathological state, neural activity segments into bursts and oscillations that produce field potentials in the range of 2-70 Hz. Electromechanical activity may also be recorded from the limbs that might signify different states of tremor, akinesia and rigidity requiring different treatment modes. BIONs with accelerometers and EMG recording capability in the limbs might be useful (as described by Loeb et al., 2001, Medical Engineering and Physics 23:9-18, and incorporated herein by reference), but would probably require rechargeable battery-power and E-field data transmission to avoid encumbering the limbs.

Site searching may be conducted by various methods known to those skilled in the art. For example, electrodes may be inserted through a rigid 2 mm guide-tube that is placed initially according to stereotaxic coordinates. A straight microelectrode probe may be passed through the guide-tube to record from the various nuclei of the BG, whose characteristic patterns of single unit activity allow them to be identified individually. Glass-insulated tungsten probes, which are made from coarsely sharpened 300μ wire with tip exposures of 10-50μ, may be utilized. The insulation and tip materials may not support extensive trial stimulation through the tips, so a second stimulation contact may be used about 2 mm proximal from the recording tip. In cases where sites can be probed only along this single depth axis, a suitable site may be found by insertion of a second guide tube and similar probing along a track ˜2 mm away and parallel to the original track. Such probes may be used instead of or in addition to the shaft 62 with both straight microelectrode 70 and lateral microelectrode 75 illustrated in FIG. 1.

The devices can be implanted and used in various ways as known by those skilled in the art. For example, various methods and devices used for implantation and use of brain stimulators are described in the following U.S. patents, which are incorporated by reference: U.S. Pat. Nos. 6,324,433 to Errico; 6,782,292 to Whitehurst; 6,427,086 to Fischell et al.; 6,788,975 to Whitehurst et al.; 6,263,237 to Rise; and 6,795,737 to Gielen et al.

Various power systems known to those skilled in the art may be used with the deep brain stimulation devices. Currently available systems use considerable power for the continuous, high frequency stimulation, which is provided by primary batteries in a hermetic package. Leads can be tunneled under the scalp and across the neck to supraclavicular site used for pacemakers. If both sides of the brain are implanted, two such leads may be connected to the stimulator. It is feasible and often necessary to have the patient awake during the electrode implantation and testing, but the tunneling requires general anesthesia, either at the end of an already lengthy surgery or as a separate surgical procedure a week or so after electrode implantation. A rechargeable lithium ion battery with disk or half-disk shape may be used. The battery may be able to power the implant for several days and be recharged enough times so that the electronics package does not have to be replaced for >10 yr.

The previous description of the disclosed embodiments is provided to enable any person skilled in the art to make or use the deep brain stimulators, methods and systems. Various modifications to these embodiments will be readily apparent to those skilled in the art, and the generic principles defined herein may be applied to other embodiments without departing from the spirit or scope of the deep brain stimulators, methods and systems. Thus, the deep brain stimulators, methods and systems are not intended to be limited to the embodiments shown herein but are to be accorded the widest scope consistent with the principles and novel features disclosed herein.

1. An implantable probe assembly that can be temporarily implanted in the brain of a patient to record and/or stimulate electrical signals in order to identify a target site for therapeutic intervention, comprising:
a) an elongated shaft;
b) an advancer attached to the elongated shaft at one end;
c) at least one electrode placed within the shaft configured to allow a target site within the brain to be identified, wherein the at least one electrode is substantially curved and is movable by the advancer while positioned within the brain.

2. The probe assembly of claim 1, further comprising a plurality of electrodes within the elongated shaft.

3. The probe assembly of claim 2, further comprising a fixed, substantially straight electrode within the shaft configured to record and/or stimulate tissue of the brain.

4. The probe assembly of claim 1, wherein the curved electrode comprises a flexible metal.

5. The probe assembly of claim 4, wherein the metal comprises iridium.

6. The probe assembly of claim 1, wherein the curved electrode is insulated with an elastic dielectric coating.

7. The probe assembly of claim 6, wherein the dielectric coating comprises one or more polymers from the family of polyparaxylylenes.

8. The probe assembly of claim 6, wherein the dielectric coating is removable from the tip of the curved electrode by laser ablation.

9. A deep brain probe system to identify a target site in the brain of a patient for therapeutic intervention, comprising:
a) a probe configured for implantation within the brain of the patient;
b) at least one electrode positioned within the probe that extends outside of the probe laterally relative to the probe's longitudinal axis, wherein the at least one electrode is substantially curved and configured to detect field potentials from the brain; and
c) a recorder configured to record data representative of the detected field potentials.

10. The system of claim 9, further comprising a plurality of electrodes, within the shaft to identify a target site for therapeutic intervention.

11. The system of claim 10, further comprising a fixed, substantially straight electrode configured so as to allow the electrode to aid in the identification of a target sight along the probe's longitudinal axis.

12. The system of claim 12, wherein the probe further comprises an advancer configured so as to be able to retract and/or advance the curved electrode while within the brain.

13. The system of claim 9, wherein the probe is configured so that it may rotate around its longitudinal axis, thereby repositioning the curved electrode within the brain.

14. The system of claim 9, further comprising a hollow guide tube to facilitate insertion of the probe into the patient's brain tissue.

15. The system of claim 9, wherein the at least one electrode is further configured to stimulate tissue of the brain.
